(19) United States
(12) Patent Application Publication
(10) Pub. No.: US 2024/0358967 A1
(43) Pub. Date: Oct. 31, 2024

(54) ENDOLUMINAL INTRODUCER SHEATH WITH POLYMER LAYERS AND SUPPORT ELEMENTS THEREBETWEEN

(71) Applicant: W. L. Gore & Associates, Inc., Newark, DE (US)

(72) Inventors: James L. Goepfrich, Flagstaff, AZ (US); William D. Montgomery, Flagstaff, AZ (US)

(21) Appl. No.: 18/692,160

(22) PCT Filed: Sep. 14, 2022
(86) PCT No.: PCT/US2022/043515

§ 371 Date: Mar. 14, 2024

Related U.S. Application Data
(60) Provisional application No. 63/244,688, filed on Sep. 15, 2021.

(51) Int. Cl. A61M 25/00

(52) U.S. Cl. CPC A61M 25/0045; A61M 25/0012; A61M 25/005; A61M 25/0054; A61M 2205/32; A61M 2207/10

(57) ABSTRACT

Endoluminal introducer sheaths and methods of forming the same are disclosed. The introducer sheath includes a first polymer layer, a second polymer layer, and at least two longitudinal support elements positioned between the first and second polymer layers. The longitudinal support elements are configured to provide columnar strength to facilitate advancement of the endoluminal introducer sheath into a vessel without substantial longitudinal compaction.

### CROSS-REFERENCE TO RELATED APPLICATION

This application is a national phase application of PCT Application No. PCT/US2022/043515, internationally filed on Sep. 14, 2022, which claims priority to U.S. Provisional Application No. 63/244,688, filed Sep. 15, 2021, which are hereby incorporated by reference in their entireties.

### FIELD

The present disclosure relates generally to medical or surgical sheaths. More specifically, the disclosure relates to sheaths with structure provided for support.

### BACKGROUND

Current methods for providing medical treatment to a human body often involve the use of catheters and medical devices that are endovascularly or endoluminally deliverable. Non-limiting examples include the endoluminal or endovascular delivery of endoprostheses, such as, for example, stents and stent grafts (self-expanding or otherwise), bifurcated stents and stent grafts, drug-eluting stents, and vascular filters, as well as endoluminal imaging devices.

Such catheters and other medical devices sometimes enter the body through an orifice or incision. In some instances, a medical conduit is inserted through the orifice or incision, and the catheter and other medical devices are passed through the medical conduit. Such medical conduits are sometimes referred to as introducer sheaths. In some cases, the introducer sheath is used to navigate through an internal lumen within the human body, during which a portion of the introducer sheath may be crimped or crushed as a result of coming into contact with a wall of the internal lumen. In such instances, it is desirable for the introducer sheath to include a structural support.

### SUMMARY

Endoluminal introducer sheaths as well as methods of manufacturing the same are disclosed.

According to one example (“Example 1”), an endoluminal introducer sheath includes a first polymer layer, a second polymer layer, and at least two longitudinal support elements positioned between the first and second polymer layers and configured to provide columnar strength to facilitate advancement of the endoluminal introducer sheath into a vessel without substantial longitudinal compaction. The longitudinal support elements are isolated from each other by the first and second polymer layers.

According to another example (“Example 2”), further to Example 1, the at least two longitudinal support elements are separate and disconnected from each other.

According to another example (“Example 3”), further to Example 1 or 2, the at least two longitudinal support elements include three longitudinal support elements.

According to another example (“Example 4”), further to Example 1 or 2, the at least two longitudinal support elements include four longitudinal support elements.

According to another example (“Example 5”), the endoluminal introducer sheath of any preceding Example, the at least two longitudinal support elements are positioned symmetrically about a circumference of the endoluminal introducer sheath.

According to another example (“Example 6”), further to any one of Examples 1-4, the at least two longitudinal support elements are positioned asymmetrically about a circumference of the endoluminal introducer sheath.

According to another example (“Example 7”), further to any preceding Example, the at least two longitudinal support elements are generally aligned with respect to a longitudinal axis of the endoluminal introducer sheath.

According to another example (“Example 8”), further to any preceding Example, the endoluminal introducer sheath has an average wall thickness of less than about 1 mm.

According to another example (“Example 9”), further to any preceding Example, at least one of the first polymer layer or the second polymer layer comprises a plurality of sublayers.

According to another example (“Example 10”), further to Example 9, at least one of the at least two longitudinal support elements is positioned between two neighboring layers of the plurality of sublayers.

According to another example (“Example 11”), further to any preceding Example, the first polymer layer is at least partially bonded to the second polymer layer, the first and second polymer layers completely encapsulating the at least two longitudinal support elements.

According to another example (“Example 12”), further to any preceding Example, further includes an intermediate layer positioned between the first and second polymer layers, wherein the first and second polymer layers are at least partially bonded to the intermediate layer.

According to another example (“Example 13”), further to any preceding Example, at least one of the at least two longitudinal support elements is at least partially radiopaque.

According to another example (“Example 14”), further to any preceding Example, the at least two longitudinal support elements are portions of at least one continuous support member.

According to another example (“Example 15”), further to any preceding Example, the at least two longitudinal support elements further include a plurality of flex points positioned along a length of the at least two longitudinal support elements.

According to another example (“Example 16”), further to any preceding Example, at least one of the first polymer layer or the second polymer layer includes at least one radiopaque mark on a surface thereof.

According to another example (“Example 17”), a method of forming an endoluminal introducer sheath includes positioning a first polymer layer around a mandrel, applying a first heat treatment to the first polymer layer, positioning at least two longitudinal support elements on the first polymer layer. The longitudinal support elements are isolated from each other by the first and second polymer layers, and the longitudinal support elements provide columnar strength to facilitate advancement of the endoluminal introducer sheath into a vessel without substantial longitudinal compaction, positioning a second polymer layer around the first polymer layer and the at least two longitudinal support elements, and applying a second heat treatment to the first polymer layer, the second polymer layer, and the at least two longitudinal support elements positioned between the first and second polymer layers.

According to another example (“Example 18”), further to Example 17, the method further includes positioning, prior to positioning the second polymer layer around the first polymer layer, an intermediate polymer layer around the first polymer layer, the intermediate polymer layer configured to be positioned between the first and second polymer layers.

According to another example (“Example 19”), further to Example 17 or 18, the method further includes applying, prior to positioning the at least two longitudinal support elements on the first polymer layer, a third heat treatment to the at least two longitudinal support elements to impart form on the at least two longitudinal support elements.

According to another example (“Example 20”), further to any one of Examples 17-19, the method further includes applying at least one radiopaque mark on a surface of at least one of the first polymer layer or the second polymer layer.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a cross-sectional side view of an endoluminal introducer sheath in accordance with an embodiment disclosed herein;

FIGS. 2A through 2E are cross-sectional front views of an endoluminal introducer sheath in accordance with embodiments disclosed herein;

FIGS. 3A through 3D are cross-sectional front views of an endoluminal introducer sheath in accordance with embodiments disclosed herein;

FIGS. 4A and 4B are cross-sectional side views of a portion of an endoluminal introducer sheath in accordance with an embodiment disclosed herein;

FIG. 5 is a flowchart of a method of manufacturing the endoluminal introducer sheath in accordance with an embodiment disclosed herein; and

FIG. 6A through 6D are elevated views of an endoluminal introducer sheath with support elements in accordance with an embodiment disclosed herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “without substantial longitudinal compaction” is used to refer to a physical property of a material or component which is capable of withstanding a certain range of external force applied to it while substantially maintaining its original longitudinal length. In some examples, the substantially maintaining of the original longitudinal length may involve maintaining at least about 75%, 80%, 85%, 90%, 95%, or 99% or more of the original longitudinal length, or any other range or value therebetween.

The term “generally aligned” with respect to a line such as an axis is used to refer to a physical positioning of an object with respect to the defined line or axis. For example, an object is aligned with an axis if the object is parallel to the axis, and the object is generally aligned with the axis if a substantial portion of the object, for example at least about 80%, 85%, 90%, 95%, or 99% or more of the object, is substantially aligned with the axis, where to be substantially aligned with the axis indicates the object positioned at an angle that is less than about 10°, less than about 5°, or otherwise within a suitable angle with respect to the axis to which it is compared.

### Description of Various Embodiments

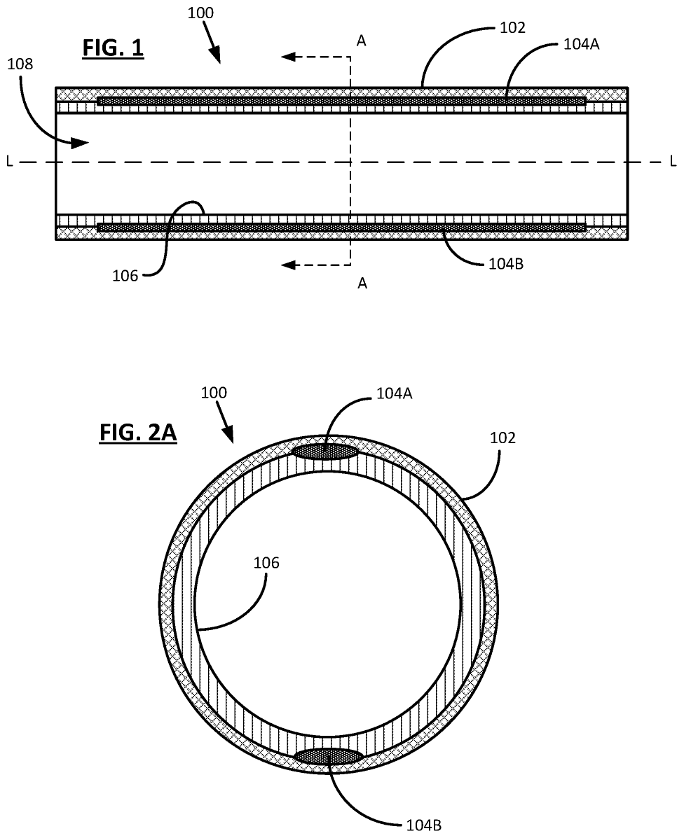

FIG. 1 illustrates a cross-sectional view of a construct 100 which may be in the shape of a tube or sheath, particularly an endoluminal introducer sheath, as disclosed herein. The construct 100 includes a first or outer layer 102, a plurality of longitudinal support elements or members 104, and a second or an inner layer 106. The inner layer 106 is also referred to as a base layer. In various examples, each longitudinal support element 104 is isolated relative to the other longitudinal support elements 104 by the first (outer) layer 102 and the second (inner) layer 106 or, if one or more of the layers 102 and 106 is made of a plurality of smaller or thinner layers (generally referred to herein as “sublayers”) (e.g., by two of the neighboring sublayers), as further explained herein. In some examples, the longitudinal support elements 104 are separate and disconnected from each other.

The outer layer 102 is at least partially attached or bonded to the inner layer 106 directly or indirectly such that the support elements 104 are completely surrounded or encapsulated by the outer layer 102 and the inner layer 106 and therefore movement of the support elements 104 with respect to the two layers 102 and 106 are reduced or limited. The attaching or bonding may be achieved via a suitable bonding method or combination of methods, such as by partially melting portions of and thereby bonding together the first and second layers 102, 106, or by the use of an adhesive, for example a heat-activated adhesive. In some examples, the support elements 104 extend a portion of the length of the construct 100. In some examples, at least one of the support elements 104 may be partially or entirely radiopaque.

In some examples, the wall of the construct 100 has an average thickness of less than about 5 mm, 2 mm, 1 mm, 0.5 mm, 0.2 mm or any other suitable range therebetween. The wall thickness is defined by the outer layer 102 and the inner layer 106. In some examples, further explained herein, the wall thickness may be defined by a plurality of layers in addition to the aforementioned layers, or the aforementioned layers being formed of a plurality of smaller or thinner layers, also referred to as “sublayers”.

The inner layer 106 defines the shape and size of a lumen or conduit 108 extending through the construct 100. The support elements 104 may be in any suitable number, for example two, three, four, or more. In the example shown, the construct 100 includes two (2) support elements, labeled 104A and 104B in the figure. In some examples, the support elements may be made of same or similar material of the same or similar physical properties. In some examples, the support elements may be made of different materials with different physical properties. In some examples, described further below, a plurality of support elements may be equally spaced apart circumferentially about the construct, or one or more of the plurality of support elements may have different spacing between adjacent support elements.

The support elements 104 are positioned between the outer layer 102 and the inner layer 106 such that the support elements 104 provide sufficient columnar strength to allow the construct 100 to be advanced into a vessel, for example that of a patient, without substantial longitudinal compaction. As such, the support elements 104 are made of a material that is less flexible or prone to longitudinal compaction than the outer layer 102 and the inner layer 106 but also capable of changing the configuration of the construct 100, for example, via bending or twisting but not crimping or crushing, thereby substantially maintaining the structural integrity of the construct 100 and the longitudinal length of the construct 100, which is the length of the construct 100 measured along a longitudinal axis L-L of the construct 100.

In some examples, sufficient column strength is determined using stress-strain test or compression force testing to determine the material's susceptibility to buckle, crack, or experience other damages. In some examples, sufficient column strength is defined by the support elements' capability of maintaining at least about 80%, 85%, 90%, 95%, or 99% of the original longitudinal length, or any other range or value therebetween, in response to an applied force representative of operating conditions, although a variety of values are contemplated. Regardless of any specific values, the column strength may generally be characterized as sufficient to prevent or substantially inhibit unwanted crumpling or collapse during axial displacement within a vessel, while still being sufficient flexible to track the tortuous vascular anatomy without significant damage to the vessel or device. In some examples, the column strength may be adjusted or determined based on the type and/or material of the polymeric layer(s) and/or longitudinal support elements or members of the construct. In some examples, as disclosed further herein, the column strength may be adjusted by controlling the heat treatment process of the polymeric layer such that the layer reaches the desired physical properties. In some examples, the column strength may be determined based on the size, thickness, and/or cross-sectional area/dimensions of the layer and/or the longitudinal support elements or members of the construct.

In some examples, the support elements are flat wires where the width is greater than the thickness of the wires. In some examples, the support elements 104 are round wires or wires with other cross-sectional shapes such as square, rectangular, ovular, or polygonal. In some examples, the support elements are substantially straight and extend along the direction defined by the longitudinal axis of the construct. In some examples, the support elements have a curved or crooked configuration but are still generally aligned with the longitudinal axis of the construct.

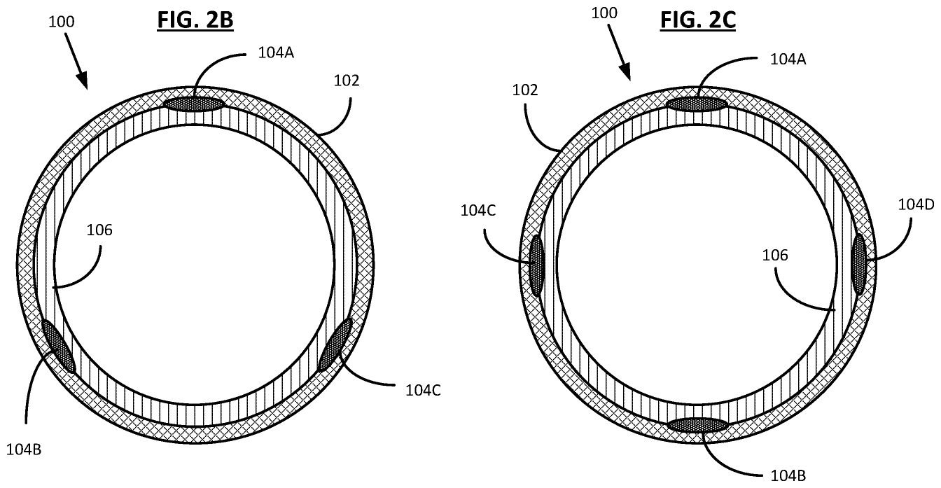

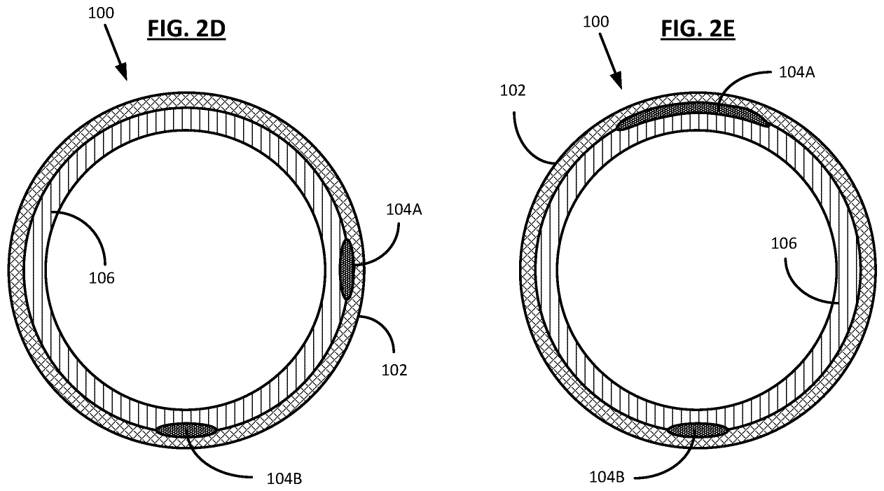

FIG. 2A illustrates the construct 100 as viewed in the direction of the arrow of line A-A in FIG. 1 according to one example, where the line A-A dissects the construct 100 perpendicularly with respect to the longitudinal axis L-L to show the cross-section. The support elements 104A and 104B may be positioned at opposite locations from each other in the construct 100 such that the support elements are located approximately 180° apart from each other.

FIG. 2B illustrates the construct 100 in another example, where the construct 100 has three (3) support elements 104A, 104B, and 104C. The support elements may be positioned so as to be equidistantly dispersed along the perimeter of the construct 100 such that the support elements are located approximately 120° apart from each other, for example, although a variety of other angular displacements are contemplated.

FIG. 2C illustrates the construct 100 in another example, where the construct 100 has four (4) support elements 104A, 104B, 104C, and 104D. The support elements may be positioned such that one pair of support elements is positioned at opposite locations from each other, and another pair of support elements is positioned at opposite locations from each other in different locations from the first pair of support elements. As such, in some examples, the support elements are located approximately 90° apart from each other. There may be additional implementations of the construct 100 with more than four (4) support elements.

FIGS. 2A through 2C illustrate examples where the support elements are positioned symmetrically (reflectional symmetry or rotational symmetry, for example) around a periphery or circumference of the construct 100. FIG. 2D illustrates an example where the support elements are positioned asymmetrically around the periphery or circumference of the construct 100. Specifically, the support elements 104A and 104B are positioned such that the distance between the support elements measured along the circumference of the construct 100 is shorter on one side than on the other side, thereby causing asymmetry in such configuration.

FIG. 2E illustrates another example where the support elements are asymmetric with respect to each other, where one support element 104A has a different shape or size from the other support element 104B. The support element 104A, for example, may have a wider or narrower width, longer or shorter length, or a generally different shape or configuration than the other support element 104B.

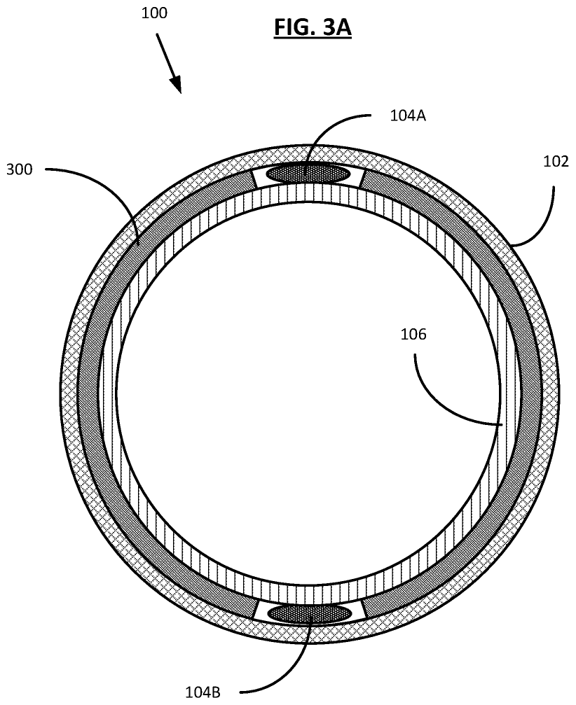

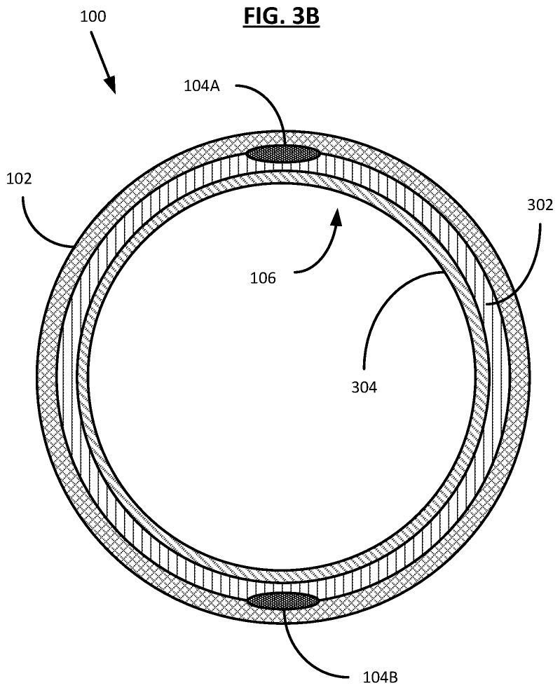

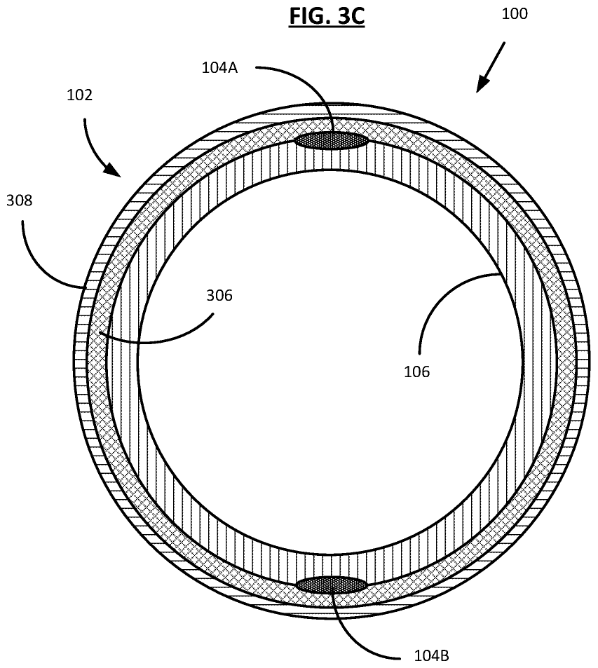

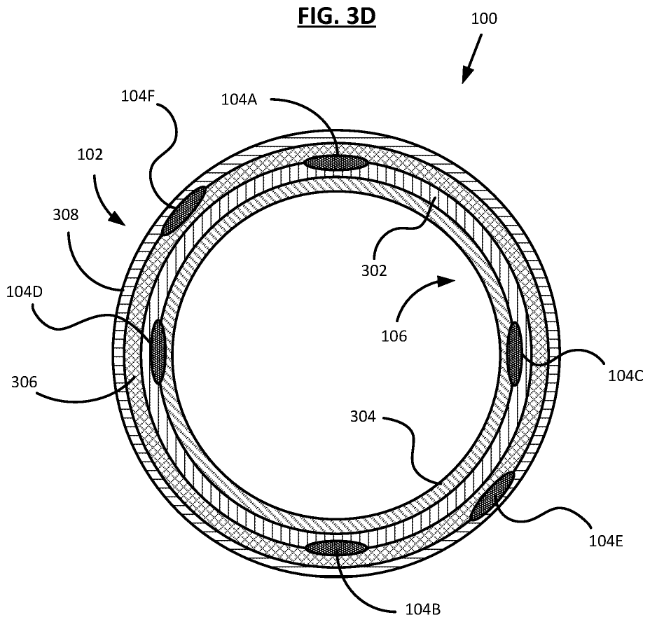

FIG. 3A shows an intermediate layer 300 between the outer layer 102 and the inner layer 106 such that the intermediate layer 300 provides the ability to adhesively or cohesively bond the outer layer 102 with the inner layer 106. An outer surface of the intermediate layer 300 at least partially attaches to an inner surface of the outer layer 102 and an inner surface of the intermediate layer 300 at least partially attaches to an outer surface of the inner layer 106. In some examples, the support elements 104A and 104B may come into contact with the outer layer 102, the intermediate layer 300, and the inner layer 106. In some examples, the intermediate layer 300 is made of any suitable heat-activated adhesive material such as any suitable types of thermoset polyester film or thermoplastic film including but not limited to fluorinated ethylene propylene (FEP), for example.

Although the layers 102 and 106 are labeled “outer layer” and “inner layer,” respectively, in some examples, each of these layers is not made of a single layer or a single sheet of material, but rather includes a plurality of layers, which may be referred to as sublayers, which are attached together to collectively form the layer.

For example, FIG. 3B shows the inner layer 106 having two sublayers 302 and 304. The first sublayer 302 is in contact with the support elements 104A and 104B as well as the outer layer 102, but the second sublayer 304 is in contact only with the first sublayer 302. The sublayers 302 and 304 are attached together, via adhesion or cohesion, for example, to form the inner layer 106. In some examples, the second sublayer 304 may be made of a different material from the first sublayer 302.

FIG. 3C, for example, shows the outer layer 102 having two sublayers 306 and 308, where the first sublayer 306 is in contact with the support elements 104A and 104B as well as the inner layer 106, but the second sublayer 308 is in contact only with the first sublayer 306. The sublayers 306 and 308 are attached together, via adhesion or cohesion, for example, to form the outer layer 102. In some examples, the second sublayer 308 may be made of a different material from the first sublayer 306.

In some examples, the outer layer 102 and the inner layer 106 each has two sublayers, forming a construct with a total of four layers. In some examples, the outer layer 102 and/or the inner layer 106 may have more than two sublayers each, for example three or more sublayers. The sublayers may be made of the same or different materials, have the similar or different physical and/or chemical properties, and/or have the same or different thicknesses.

FIG. 3D, for example, shows a plurality of support elements 104A through 104F, each positioned between two neighboring sublayers, and each of the outer layer 102 and the inner layer 106 has two sublayers, forming a total of four sublayers 302, 304, 306, and 308. The support elements 104A and 104B are both positioned between the sublayer 302 and the sublayer 306. The support elements 104C and 104D are both positioned between the sublayer 302 and the sublayer 304. The support elements 104E and 104F are both positioned between the sublayer 306 and the sublayer 308. Other additional or alternative layers or sublayers may also be implemented as suitable.

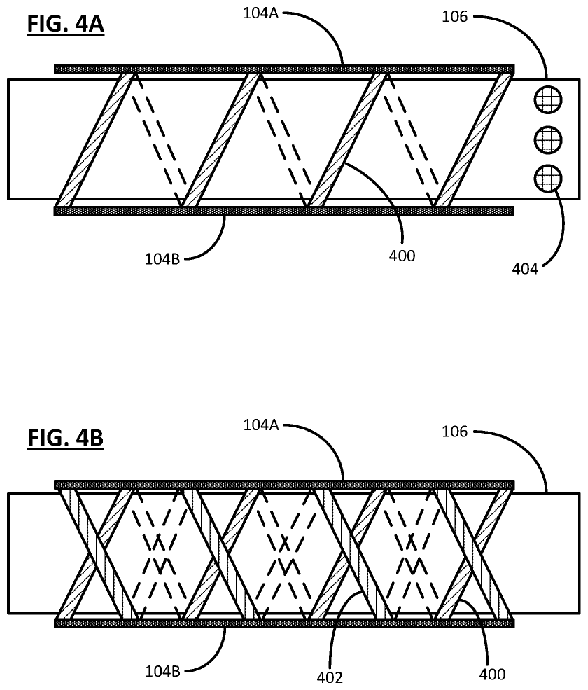

FIG. 4A shows an additional support element 400 which may be implemented with the support elements 104A and 104B. The additional support element 400 may be a helical member wrapped around the inner layer 106 so as to provide radial support in addition to the longitudinal support provided by the support elements 104A and 104B. In some examples, the support element 400 may be attached to the support elements 104A and 104B at locations where the support elements come into contact with each other. In some examples, the support element 400 may be a continuous extension of the support element 104A or 104B, for example cut (e.g., via laser cutting) from a continuous or monolithic tubular piece of material.

FIG. 4B shows two additional support elements 400 and 402 which may be implemented with the support elements 104A and 104B. The support elements 400 and 402 may be helical members wrapped around the inner layer 106 such that the first helical support element 400 is wound at a different angle from the second helical support element 402, for example. The support elements 400 and 402 may be attached to the support elements 104A and 104B at locations where the support elements come into contact with each other.

In some examples, radiopacity may be implemented on one or more layers or sublayers of the construct or sheath as disclosed herein. For example, FIG. 4A shows radiopaque marks 404 which may be disposed on an inner surface and/or an outer surface of the inner layer 106. The marks 404 allow the user (e.g., surgeon or physician) to see the location of the construct while the construct is inside the patient when X-ray or similar radiation is applied, for example. It should be understood that the marks 404 may be applied in any number at any location on any layer or sublayer in any one or more of the embodiments as disclosed herein. In some examples, the marks 404 may be formed on an inner or outer surface of a sublayer such that the marks 404 are located between two neighboring layers or sublayers. In some examples, multiple marks 404 may be disposed between multiple different pairs of neighboring layers or sublayers. In some examples, the different marks 404 disposed on different locations may have different and distinct shape or configuration to be distinguishable from one another. In some examples, the marks 404 may be applied on any suitable outer or inner surface using a pad printing method. The marks 404 may take the form of dots, polygons, lines, geometric patterns, or any other suitable configuration that allows for easy recognition by the user during use. The marks 404 may be made of any suitable radiopaque material including, but are not limited to, tungsten.

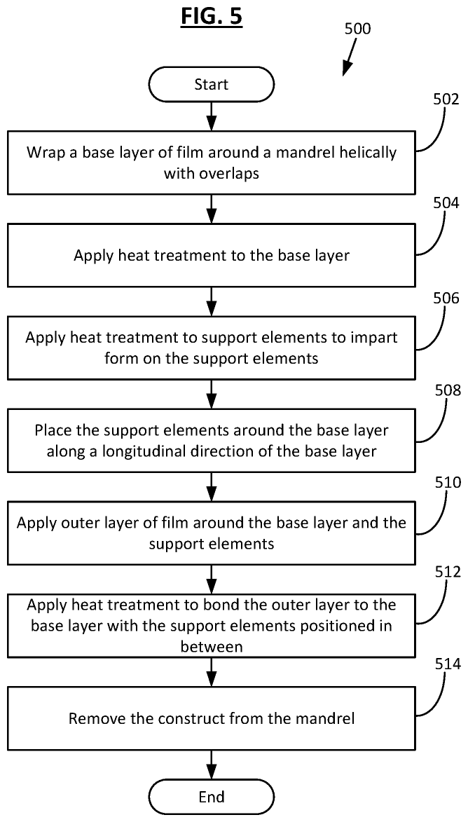

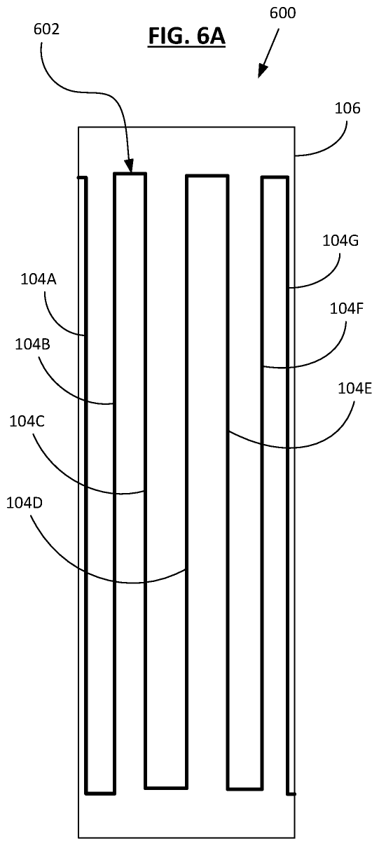

FIG. 5 shows a process 500 by which the construct 100 may be made or manufactured, according to embodiments disclosed herein. In step 502, the material for the base layer (e.g., a strip of polymer such as a film) is wrapped or positioned around a mandrel. The material, or film, may be wrapped helically such that a portion of the material in one turn of the helix overlaps with another portion of the material in a subsequent turn of the helix, thereby forming the overlapped helical configuration.

In step 504, a heat treatment is applied to the base layer while the base layer is wrapped around the mandrel. The heat treatment may involve any suitable time and temperature to heat the material of the base layer without reaching its melting point, then cooling the base layer in a controlled way to select the desired physical properties of the base layer.

In step 506, a separate heat treatment is applied to the support elements. The heat treatment in this step has different temperature and treatment time from the heat treatment of step 504, and the heat treatment is configured to impart form on the support elements in order to select the desired physical and mechanical properties of the support elements.

In step 508, the longitudinal support elements are placed or positioned around the base layer along a longitudinal direction of the base layer. The longitudinal support elements may be separate and disconnected from each other. The longitudinal direction may be defined by the mandrel. The support elements are positioned such that they are generally aligned with, or substantially parallel to, the longitudinal axis of the base layer or the mandrel.

In step 510, another layer of film, also referred to as a second layer of film, is wrapped, applied, or positioned around the base layer and the support elements to form the outer layer. In some examples, the film is wrapped in an overlapping helical configuration, similar to step 502. In some examples, the film has a different wrapping angle or number of turns from the helical configuration assumed by the first layer of film in step 502. In some examples, the thickness or width of the second layer of film may be different from the first layer of film which forms the base layer.

In step 512, a heat treatment is applied in order to bond the second layer of film forming the outer layer to the first layer of film forming the base layer, along with the support elements positioned therebetween. As such, each longitudinal support elements may be isolated from the rest of the longitudinal support elements by the first (outer) layer and the second (inner) layer when bonded together, or otherwise isolated from one another. In some examples, the heat treatment temperature is selected to partially melt a portion of the first or second layer of film so as to bond to the other layer of film. In some examples, a surface of the first or second layer of film is treated with a heat-activated adhesive as known in the art, which is kept at a certain temperature for a period of time for the adhesive to activate. The heat treatment thus activates the adhesive, bond the two layers together, and subsequently crystallizes when the adhesive cools down, thereby increasing the strength of the material bond.

In step 514, which occurs after the cooling of the layers is observed, the product, or the construct, is removed from the mandrel.

In some examples, either before or after step 508, an intermediate layer of film may also be placed or positioned around the base layer. The intermediate layer may include a heat-activated adhesive to facilitate bonding the two surrounding layers together as explained in step 512. In some examples, step 502 and/or step 510 includes applying a plurality of layers of film which may be made of the same material or different materials, as suitable.

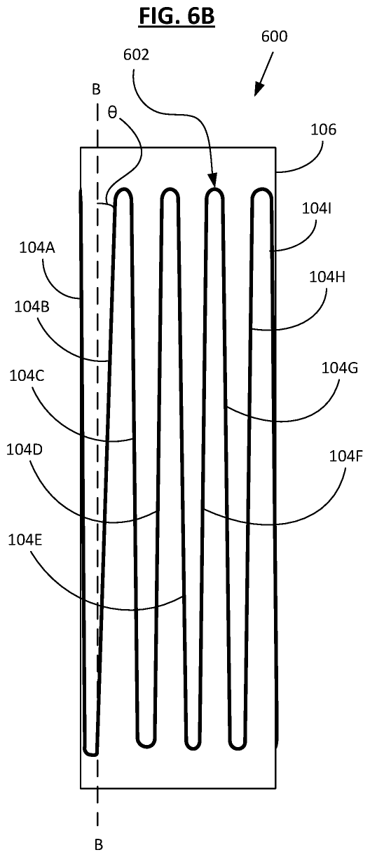

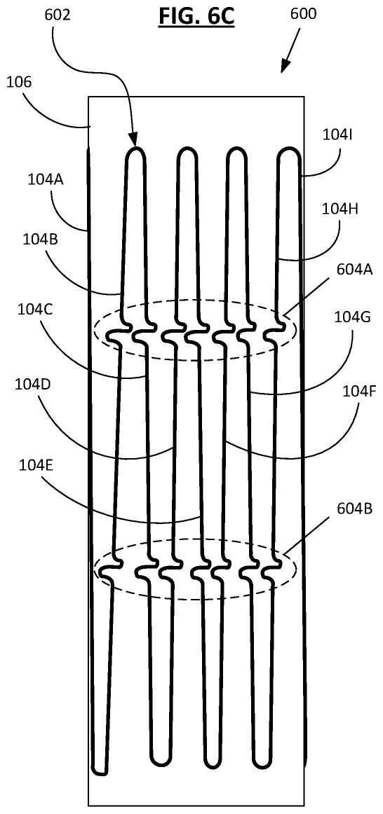

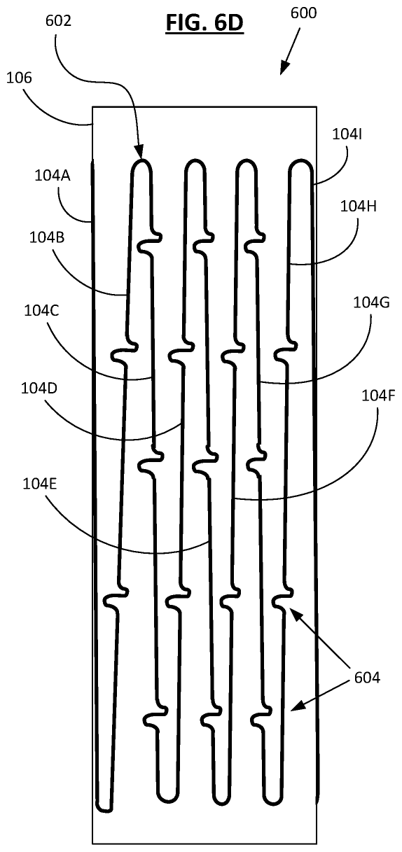

FIGS. 6A through 6D show a continuous support member 600, which is made of a plurality of longitudinal support elements 104 connected together via a plurality of connection apices 602, according to various examples as disclosed herein. As such, according to some examples, the longitudinal support elements 104 form, or are portions of, the continuous support member 600. In some examples, the continuous support member 600 is made of a continuous and unitary piece of material. For reference, the outer layer 102 is not shown for simplicity. In various examples, each of the apices 602 connects two neighboring longitudinal support elements 104 to form a continuous support member 600. The support member 600 may include any number of individual longitudinal support elements 104 as suitable, and the shape and configuration of the apices 602 may vary, for example being substantially straight, curved, or crooked. In some examples, there may be a plurality of continuous support members 600, each separate from one another, positioned around the inner layer 106. In some examples, the longitudinal length of the continuous support member 600 measured along the length of the inner layer 106 may be different from the longitudinal lengths of the other continuous support members 600 positioned around the inner layer 106.

In FIG. 6A, the continuous support member 600 assumes a configuration similar to an elongated square wave, showing at least seven individual support elements 104A through 104G extending along a longitudinal length of the inner layer 106 in a direction substantially parallel with the longitudinal axis L-L as shown in FIG. 1. In some examples, each of the individual longitudinal support elements 104A through 104G may be sufficiently or effectively parallel with respect to each other. In some examples, the square wave configuration may resemble a trapezoidal shape such that each of the longitudinal support elements 104A through 104G may be slanted with respect to each other.

In FIG. 6B, the continuous support member 600 assumes a configuration which may be described as serpentine, rounded zigzag, undulating, or elongated sinusoidal, showing at least nine individual support elements 104A through 104I, for example. As shown, the apices 602 may be substantially curved or rounded, and each of the individual support elements 104A through 104I is slightly slanted such that it assumes an angular position with respect to an axis parallel to the longitudinal axis L-L of the construct.

For example, the individual support element 104B is shown to extend at an angle (θ) with respect to a line B-B which extends parallel to the longitudinal axis L-L of the construct, where the value of θ may be any nonzero value less than approximately 20°, less than approximately 15°,less than approximately 10°, less than approximately 5°, less than approximately 3°, less than approximately 1°, or any other value therebetween, although a variety of additional angular values are contemplated. Similarly, any one or more of the other individual support elements may also be positioned at an angle which may be the same as or different from the angle (θ) of the support element 104B.

As shown in FIG. 6C, the continuous support member 600 further includes at least one flex point 604 along the length of at least one of the individual longitudinal support elements 104. The flex point(s) 604 may be sinusoidal, undulating, or zigzag in configuration in a direction different from the direction in which the longitudinal support elements 104 extend. In some examples, the flex points 604 may each assume an S-shape, a reverse S-shape, a Z-shape, or a reverse Z-shape when viewed along the longitudinal axis of the construct. In some examples, the flex points 604 may be extending transversely or perpendicularly with respect to the longitudinal axis of the construct. In some examples, the flex points 604 are aligned along certain points or locations along the length of the longitudinal support elements 104, such that the flex points 604 are positioned along a straight line across a surface of the inner layer 106.

In some examples, there may be multiple sets of flex points 604 (for example, a first set 604A and a second set 604B as shown) such that all of the flex points belonging to a set is positioned along a straight line drawn across the surface of the inner layer 106. In some examples, the two sets 604A and 604B are substantially parallel with respect to each other, and/or they extend substantially perpendicularly with respect to the longitudinal axis. In some examples, where there are more than two sets of flex points 604, the multiple sets may be positioned equidistantly along the longitudinal axis of the construct, or they may alternatively be positioned such that a first distance between a first set and a second set may be different from a second distance between the first set and a third set, where the second and third sets are positioned on both sides of the first set with no other set in between.

As shown in FIG. 6D, the flex points 604 may be positioned in a staggered configuration with respect to each other, where the locations of the flex points 604 are dispersed throughout the length of the continuous support member 600. In some examples, the flex points 604 are positioned equidistantly along the length of the continuous support member 600 such that two neighboring flex points 604 share the same distance along the continuous support member 600 when the continuous support member 600 is laid out in a straight line (that is, when the apices 602 are unbent), instead of along the longitudinal axis.

As disclosed herein, the support elements and/or the continuous support members may be made of any suitable material such as nitinol (NiTi) and/or other materials such as, but not limited to, stainless steel, L605 steel, polymers, MP35N steel, polymeric materials, Phynox, Elgiloy, or any other appropriate biocompatible material, and combinations thereof, can be used as the material of the support elements. The super-elastic properties and softness of NiTi may enhance the conformability of the support elements. In addition, NiTi can be shape-set into a desired shape. That is, NiTi can be shape-set so that the support elements tend to turn into a desired shape when the support elements are unconstrained, such as when the support elements are deployed out from a delivery system.

In some examples, the support elements may be made of any suitable radiopaque material including but not limited to platinum or titanium, among others.

As disclosed herein, any of the layers or sublayers may be made of polymer or polymers. For example, a biocompatible material including but not limited to a fluoropolymer, such as a polytetrafluoroethylene (PTFE) polymer or an expanded polytetrafluoroethylene (ePTFE) polymer may be used. In some examples, the biocompatible material used in the implant may include, but not limited to, polyethylene or expanded polyethylene.

In some instances, materials such as, but not limited to, polyester, silicone, urethane, polyethylene terephthalate, another biocompatible polymer, or combinations thereof, may be used to form at least a portion of the layers or sublayers. In some instances, bioresorbable or bioabsorbable materials may be used, for example a bioresorbable or bioabsorbable polymer. In some instances, the materials can include Dacron, polyolefins, carboxy methylcellulose fabrics, polyurethanes, or other woven, non-woven, or film elastomers.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

The devices, methods, and systems shown in the figures disclosed herein are provided as examples of the various features of the devices, methods, and systems and, although the combination of those illustrated features is clearly within the scope of invention, the examples and their illustrations are not meant to suggest the inventive concepts provided herein are limited from fewer features, additional features, or alternative features to one or more of those features shown in the figures.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An endoluminal introducer sheath comprising:
a first polymer layer;
a second polymer layer; and
at least two longitudinal support elements positioned between the first and second polymer layers and configured to provide columnar strength to facilitate advancement of the endoluminal introducer sheath into a vessel without substantial longitudinal compaction, wherein the longitudinal support elements are isolated from each other by the first and second polymer layers.

2. The endoluminal introducer sheath of claim 1, wherein the at least two longitudinal support elements are separate and disconnected from each other.

3. The endoluminal introducer sheath of claim 1, wherein the at least two longitudinal support elements include three longitudinal support elements.

4. The endoluminal introducer sheath of claim 1, wherein the at least two longitudinal support elements include four longitudinal support elements.

5. The endoluminal introducer sheath of claim 1, wherein the at least two longitudinal support elements are positioned symmetrically about a circumference of the endoluminal introducer sheath.

6. The endoluminal introducer sheath of claim 1, wherein the at least two longitudinal support elements are positioned asymmetrically about a circumference of the endoluminal introducer sheath.

7. The endoluminal introducer sheath of claim 1, wherein the at least two longitudinal support elements are generally aligned with respect to a longitudinal axis of the endoluminal introducer sheath.

8. The endoluminal introducer sheath of claim 1, wherein the endoluminal introducer sheath has an average wall thickness of less than about 1 mm.

9. The endoluminal introducer sheath of claim 1, wherein at least one of the first polymer layer or the second polymer layer comprises a plurality of sublayers.

10. The endoluminal introducer sheath of claim 9, wherein at least one of the at least two longitudinal support elements is positioned between two neighboring layers of the plurality of sublayers.

11. The endoluminal introducer sheath of claim 1, wherein the first polymer layer is at least partially bonded to the second polymer layer, the first and second polymer layers completely encapsulating the at least two longitudinal support elements.

12. The endoluminal introducer sheath of claim 1, further comprising an intermediate layer positioned between the first and second polymer layers, wherein the first and second polymer layers are at least partially bonded to the intermediate layer.

13. The endoluminal introducer sheath of claim 1, wherein at least one of the at least two longitudinal support elements is at least partially radiopaque.

14. The endoluminal introducer sheath of claim 1, wherein the at least two longitudinal support elements are portions of at least one continuous support member.

15. The endoluminal introducer sheath of claim 1, the at least two longitudinal support elements further comprising a plurality of flex points positioned along a length of the at least two longitudinal support elements.

16. The endoluminal introducer sheath of claim 1, wherein at least one of the first polymer layer or the second polymer layer includes at least one radiopaque mark on a surface thereof.

17. A method of forming an endoluminal introducer sheath comprising:
positioning a first polymer layer around a mandrel;
applying a first heat treatment to the first polymer layer;
positioning at least two longitudinal support elements on the first polymer layer, wherein the longitudinal support elements are isolated from each other by the first and second polymer layers, and the longitudinal support elements are configured to provide columnar strength to facilitate advancement of the endoluminal introducer sheath into a vessel without substantial longitudinal compaction;
positioning a second polymer layer around the first polymer layer and the at least two longitudinal support elements; and
applying a second heat treatment to the first polymer layer, the second polymer layer, and the at least two longitudinal support elements positioned between the first and second polymer layers.

18. The method of claim 17, further comprising:
positioning, prior to positioning the second polymer layer around the first polymer layer, an intermediate polymer layer around the first polymer layer, the intermediate polymer layer configured to be positioned between the first and second polymer layers.

19. The method of claim 17, further comprising:
applying, prior to positioning the at least two longitudinal support elements on the first polymer layer, a third heat treatment to the at least two longitudinal support elements to impart form on the at least two longitudinal support elements.

20. The method of claim 17, further comprising:
applying at least one radiopaque mark on a surface of at least one of the first polymer layer or the second polymer layer.
